(19) United States
(12) Patent Application Publication
(10) Pub. No.: US 2025/0342593 A1
(43) Pub. Date: Nov. 6, 2025

(54) IMAGE PROCESSING

(71) Applicants: Bruce Lawrence John MURRAY, Edinburgh (GB); Nick AGER, Edinburgh (GB); Moletest Limited, Guernsey Channel Islands (GB)

(72) Inventors: Bruce Lawrence John MURRAY, Edinburgh (GB); Nick AGER, Edinburgh (GB)

(21) Appl. No.: 18/869,478

(22) PCT Filed: May 26, 2023
(86) PCT No.: PCT/GB2023/051401

§ 371 Date: Nov. 26, 2024

(30) Foreign Application Priority Data
May 26, 2022 (GB) 2207799.4

(51) Int. Cl. G06T 7/00; A61B 5/00; G06T 3/40; G06T 7/174; G06T 11/00

(52) U.S. Cl. CPC G06T 7/0014; A61B 5/444; G06T 3/40; G06T 7/174; G06T 11/00; G06T 2207/20016; G06T 2207/30088; G06T 2207/30096; G06T 2210/36; G06T 2210/41

(57) ABSTRACT

Machine-readable instructions for execution by a data processor, for processing a captured image which includes a lesion to determine a boundary of the lesion, which instructions are arranged to display an the image, which includes a lesion under investigation and includes a portion of skin adjacent to the lesion, determine a lesion skin reference tone within the image and determine an adjacent skin reference tone of skin adjacent to the lesion which is also in the image, which comprises selecting one or more representative pixels of the lesion and not o f the lesion; analysing tone levels of pixels within the image to determine said pixels of the image as either lesion tone pixels or adjacent skin tone pixels; and determining within the image a boundary of where lesion skin tone pixels meet adjacent skin tone pixels.

### TECHNICAL FIELD

The present invention relates generally to image processing and data acquisition.

### BACKGROUND

It is known that images of lesions can be used by clinicians to provide an assessment of whether lesions can be excluded from being potentially malignant lesions. We have realised that improved edge detection allows for better assessment of skin lesions.

Differences between the images of melanoma and ‘not-melanoma’ images can be very subtle. Many lesions have no easily defined edge. Pigmented skin lesions are often similar in colour to the surrounding skin. Also, lesions can be extremely irregular in shape. Skin lesions can have an outer coloured ring then a darker edge and within the lesion yet another edge. This makes visual determination of an edge subjective and can cause a wide variation in evaluation by known image processing.

We have devised an improved way of determining an edge of a skin lesion.

### SUMMARY

According to a first aspect of the invention there are provided machine-readable instructions for execution by data processor, for processing an image which includes a lesion to determine a boundary of the lesion, which instructions are arranged to implement at least the following actions:
displaying the image, which image includes a lesion under investigation and includes a portion of skin adjacent to the lesion;
determining a lesion skin reference tone within the image, which comprises selecting one or more representative pixels within the lesion; determining an adjacent skin reference tone of skin adjacent to the lesion which is also in the image, which comprises selecting one or more representative pixels not of the lesion; and
determining within the image a boundary of where lesion skin tone pixels meet adjacent skin tone pixels, wherein pixels of the image are analysed for tone levels.

By ‘lesion’ we include a portion of skin that has an abnormal growth or appearance compared to the skin around it.

By ‘adjacent skin reference tone’ we include the tone of skin which is next to and/or surrounds the lesion, and/or which may be substantially free of lesions. Reference to tone may include colour, and so different colours and/or variation in colour, could be used.

The invention may be viewed as enabling data acquisition with improved accuracy.

One or more of the actions may comprise performing image processing calculations.

The instructions may comprise:
an image display module/block,
at least one skin tone analysis module/block, and
a boundary determination module/block.

The step of analysing tone levels of the pixels may comprise commencing the same from one or more pixels adjacent to the respective one or more representative pixels. One or both of the reference tones may comprise an average tonal value obtained from multiple pixels. The instructions may cause the analysis of tone levels to evolve progressively away from the respective one or more representative pixels.

The instructions may be configured to analyse pixels, individually and/or in groups, in the image and determine if they are lesion tone pixels or adjacent skin tone pixels. Such analysis may comprise determining if a detected tone level is within a respective reference tonal range, or is beyond a tonal reference value, or meets a reference tonal value (perhaps within a degree of tolerance). A calculated or predetermined tonal reference range or reference value may be used for the analysis for the adjacent skin tone and/or for the lesion tone, which may be determined, at least in part, by the skin lesion tone and/or the adjacent skin tone determined from the image.

For analysis of adjacent skin tone pixels, said progression may be towards the centre of the image. For lesion tone pixels said progression may be away from the centre of the image. These steps may be termed ‘populating’ image space, starting from the respective one or more representative pixels, and assigning (and storing) a value or indicator which is indicative of whether the pixel or pixel group is a lesion skin tone or an adjacent skin tone.

The step of determining the lesion reference skin tone may comprise determining a darkest level of lesion skin tone (as represented in the image). The step of determining a darkest level of lesion skin tone may include identifying at least one pixel from within the lesion region is determined as being the pixel from a subset which has substantially the darkest level of tone of the lesion. A selected pixel may have the darkest tone, or be a pixel which is one of multiple pixels in the region with substantially the same level of tone, which is the darkest level of tone of the pixels within said region.

The step of determining lesion skin reference tone may comprise determining a set of pixels of the lesion. The set of pixels may be at or proximal to a central region of the image. The step of determining skin lesion reference tone may include identifying a region of the lesion of predetermined pixel dimensions, or size. Said region may be of size 100×100 pixels, +/−25×25 pixels, for example. Said region may occupy a substantially central part of the image, or may overlap a central point of the image.

The step of determining an adjacent skin reference tone of skin adjacent to the lesion may include identifying at least one pixel which is away from the lesion and within the image. Said at least one pixel may be in a corner region of the image. Said step may include identifying at least one pixel in more than one corner region of the image, or may include identifying at least one pixel in each of the corner regions of the image.

The lesion skin tone may be darker than the adjacent skin tone, and the adjacent skin tone may be lighter than the lesion skin tone.

The instructions may be arranged to generate an initial determined boundary of the lesion. The instructions may be arranged to then determine a refined boundary with a higher accuracy than the initial boundary. The generation of the refined boundary may, at least in part, use or be based on the initial boundary. The generation of the refined boundary may comprise analysis of the image at a higher accuracy.

The instructions may be such as to generate a cropped and/or magnified image based on the location in the image of the (initial) boundary (which should in turn be indicative of the position of the lesion), which includes the lesion. The refined boundary may be determined based on use of this cropped/magnified image. Determination of the refined boundary may be arranged to be effected by the instructions using a higher resolution of the image (as compared to the image resolution used to determine the initial boundary).

The initial boundary and/or the refined boundary may be determined by the instructions using analysis of pixel tone values, in the manner in one or more ways set out in any of the preceding paragraphs.

The initial boundary may be determined by the instructions using a version of the captured image which is of lower resolution as compared to the captured image. Said version of the image may be less than fifty percent of the resolution as compared to that of the captured image, or may be between twenty to forty percent of the resolution of the captured image. The instructions may be arranged to generate a lower resolution version of the image as compared to the resolution of the captured image.

With the initial boundary of the lesion determined, the instructions may locate/position said initial boundary within the (originally) captured image, or within a version of the captured image which is of higher resolution than the version of the image used to determine the initial boundary (but of lower resolution than the captured image). The higher resolution need not correspond to the resolution of the captured image. This positioning/locating of the initial boundary in the higher resolution image may include the steps of scaling co-ordinates of the initial boundary (by a factor as determined by the relative sizes/resolutions of the image used for the initial boundary and the higher resolution image), and then mapping the initial boundary onto the higher resolution image.

The instructions may then be such as to generate a cropped image from the image of higher resolution, which includes the initial boundary, and which includes a margin of non-lesion skin which surrounds the lesion. The instructions may be such that the lesion occupies the majority of the cropped image, and the lesion may occupy between seventy-five to ninety percent of the cropped image. The lesion may be centred, or substantially so, within the cropped image.

The instructions may be such as to perform pixel tone analysis on the cropped image, and thereby determine a refined boundary.

This (the boundary determination phase) may be considered as a two-stage process in which a lesion edge is determined first with a lower level of accuracy and then with a higher level of accuracy.

The boundary determination module/block may comprise an initial boundary sub-module and a refined boundary sub-module.

The instructions may comprise an image capture module/block, which may allow, and/or guide, a user to take a picture of a lesion and a portion of skin surrounding the lesion.

The instructions may comprise generation of centring graphics, which may comprise two elongate linear features (such as lines, broken or solid) arranged substantially orthogonally to one another, which are arranged to be displayed to a user. The centring graphics may be termed cross-hairs. The centring graphics are preferably arranged to be displayed in a field of view display of a camera functionality. The centring graphics may overlay or be superimposed on a user-selected field view of the camera functionality, such as a view of skin which has a lesion. The instructions may prompt and enable a user to take a picture, using camera functionality, of skin having a lesion. The centring graphics may be presented to assist a user when taking a picture of the lesion and surrounding skin, and including to centre the lesion in a presented field of view, in an image capture mode enabled by the instructions. In the image capture mode, a magnified view may be displayed with the centring graphics superimposed on the view.

The instructions may be such as to direct or prompt the user to arrange the lesion substantially centrally of an image display region. This may be required prior to commencement of determining lesion skin tone and adjacent skin tone. The instructions may be such that once the user has centred the image to be taken, instructions then proceed to determine the boundary of the lesion in a substantially automated fashion.

The instructions may be configured to include the step of generating a magnified version of the image.

The instructions may be such as to output an image which includes a determined boundary and the lesion, and the determined boundary is superimposed on the image of the lesion. Said determined boundary may be the refined boundary. The instructions may be such as to prompt the user review the image, and provide an input in relation to a user's assessment of the perceived accuracy of the (calculated and) displayed boundary corresponding to the boundary of the lesion. The instructions may be such so as to require an affirmative user input to classify as being suitable for performing one or more assessments on. Otherwise, part or all of the boundary determination procedure may be re-run.

The instructions may be embodied as a software product, which may be a software application or an App. The software product may be arranged for use on/execution by a personal electronic device (PED), such as a smartphone or a tablet. More generally, the instructions are suitable for execution by a computer which comprises a data processor and a memory.

The instructions may be configured to generate a graphic user interface.

According to a further aspect of the invention there are provided instructions which when executed by a processor use a two-stage process to determine a boundary of a lesion, in which a first initial (approximate) lesion boundary is determined using pixel tone analysis, which is then applied on a higher resolution version of the image so as to determine a refined lesion boundary, also using pixel tone analysis.

According to another aspect of the invention there is provided a user device which is loaded with the machine-readable instructions of the first or further aspect of the invention.

The user device may be a personal electronic device, PED, such as a smartphone or tablet device, which comprises a camera, a display screen, a data processor and a memory. Typically such a device is portable.

Any of the aspects of the invention may include one or more features, either individually or in combination, as disclosed in the description or in the drawings. This disclosure includes that individual features or multiple features can be incorporated into any of the aspects of the invention, notwithstanding that they be disclosed as being connected or associated with other features, say in the context of a particular embodiment or entity or a sequence or combination of method or process steps.

### BRIEF DESCRIPTION OF THE DRAWINGS

Various embodiments of the invention will now be described, by way of example only, with reference to the following drawings in which:

FIG. 1 is a schematic view of a personal electronic device, PED;

FIG. 2 is enhanced representation of an image of a lesion and surrounding skin captured;

FIG. 3 is a view of a displayed image which shows a close-up view of part of the image shown in Figure, which includes centring cross-hairs;

FIG. 4 is a graphic user interface which includes an image of a lesion and surrounding skin, and a picture of the lesion in which the determined boundary is shown as overlain thereon; and

FIG. 5 is a flow diagram which shows the sequence of processing steps which is performed in determining an edge of the lesion.

### DETAILED DESCRIPTION

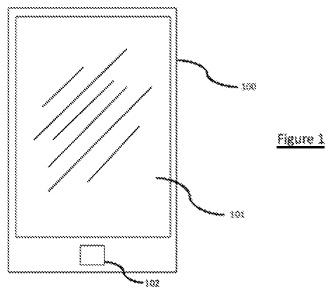

There is now described a novel system for acquiring an accurate edge of a skin lesion. A configured user device, such as a smartphone or a tablet device, which is provided with camera functionality, executes a software application, which software application comprises various functional modules which are implemented. An example of such a device 100 is shown in FIG. 1, which includes a touch screen 101 and a user input button 102. In overview, a user is guided, by displayed instructions, to take a picture of a lesion, which image is then capable of analysis so as to determine whether the malignancy of the lesion can be excluded. As is described in more detail below, this analysis comprises determining a boundary of the lesion.

To commence use of the system, the user opens the software on their PED. This then first prompts the user to direct the camera of the PED at a lesion which is to be processed. The user (i.e. the person using the system) need not necessary be the person who has the lesion.

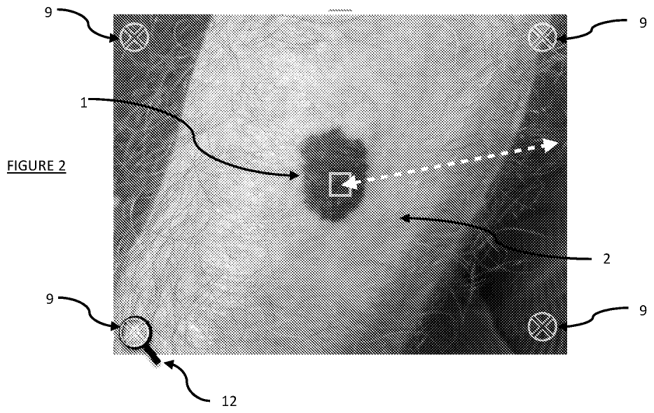

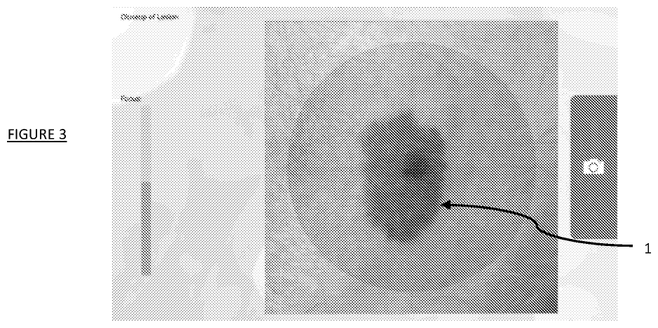

In this image capture phase, the screen of the PED shows a field of view of the camera. The task of the user is to take a picture of the both the lesion and the surrounding skin. In the example shown in FIG. 2, a lesion 5 is located on a person's forearm. The image which is taken, which is that as shown, includes both the lesion as well as the (lesion-free) skin around the lesion. In order to help to take the optimum image for the purpose of the system, a centring guide is displayed, as shown in FIG. 3. This displays a magnified image of the lesion. Text (not shown) which is displayed to the user prompts the user to centre the lesion with reference to the cross-hairs, and also ensure that the lesion is within the circular border (with the field of view display outwardly of the circular border being ‘greyed’ or partially obfuscated the field of view of the camera as displayed). The user therefore moves the PED so that the lesion is so located. When this has been achieved the user can then take the picture. The resulting picture is one in which the lesion is substantially central of the image frame, as shown in FIG. 2.

Having now captured the image, the software application begins to process the same and begins the edge determination phase. In a first part of this phase, an approximate boundary of the of lesion is determined as follows. For this initial boundary determination, a lower resolution version of the captured image is generated and used.

A sample of a set of pixels is selected from the central region of the image frame. This may be a 100×100 pixel square (as shown schematically by the square in FIG. 2). The pixels contained within that set are analysed for the darkest pixel, that is the pixel which has the darkest tone, or where there is more than one pixel within the set which have an equally darkest tone, the tone level of those pixels is stored. This may be referred to as a lesion tone reference level.

A surrounding skin tone reference level is then determined. This is done by selecting one more pixels in each of the corners of the image frame. In FIG. 2 this is depicted, for the purpose of ease of explanation, by way of icons 9. The magnifying glass icon 12 is shown to depict that the bottom left-hand corner region is being analysed. A selection of pixels from each corner is analysed for determining a lightest tone of pixel. It is a requirement that the lightest determined tone level is lighter than the reference tone level determined for the lesion. The point at which the pixels are selected in each corner is predetermined and is, for example a predetermined distance from each corner along a diagonal which connects opposite corners of the image frame. For example, a pixel one-tenth diagonally from each corner of the close-up image may be used in determining the corner regions to be analysed for the purpose of determining a reference surrounding skin tone level. In case any of the corners of the image has artefacts other than skin, or any of the corner regions does not include skin, because of limb curvature or shadow cast, each corner is analysed sequentially. The assumption is made that there is at least one corner with lighter skin for the system to work.

For the lesion edge determination process which is implemented it is required for there to be a difference in tone between the lesion and the surrounding skin in order for an edge to be detected. For the automated edge detection to operate the lesion is assumed to be darker than the surrounding skin.

Once each of the skin tone reference levels has been determined, the following image processing steps are implemented.

An initial (or approximate) determination of the boundary of the lesion is then performed. This is effected by analysing each pixel of each of the lesion region and the surrounding skin region for a level of tone as related to the reference tone levels. In this, each pixel is determined as being either a pixel depicting surrounding skin or a pixel depicting part of the lesion. This may be described as an approach whereby the two regions must preserve the contrast between their respective tonal minima. In other words, each region has a distinct range the skin much lighter than the lesion and when the growth meets a step change this contrast produces a mask representing the lesion with the surround skin made transparent. A particular starting pixel in each of the regions, such as one of the pixels which was used in determining reference tone level, can be used as the seed of a binary growth, in which each adjacent pixel is determined as being a lesion pixel or a surrounding skin pixel. Where they meet, a consistent edge is created. A flood fill algorithm may be used outwards from the central darkest pixel and inwards from the skin colour pixel. A greyscale map is generated which is flood-filled to remove any ‘holes’. The result of this is an approximate determination of the edge of the lesion based on where lesion pixels neighbour surrounding skin pixels.

A cropped image of the lesion is now generated based on the location of the just-calculated lesion edge in which the frame of the cropped image is very close to the edge. The cropped image is generated by scaling the co-ordinates of the initial boundary to a higher resolution version of the image, which may be the resolution of the image as originally captured, or a resolution which is higher than the resolution than the image used to determine the initial boundary, and mapping the initially determined boundary onto the higher resolution image. The image is then cropped so that the majority of the image includes the lesion, but nevertheless with a surrounding margin of surrounding skin.

A second stage of the edge determination phase is now effected using the cropped image. Stored reference tone levels are again used, in the same way as described above for determining the initial boundary, but at a more detailed level (since a higher resolution image is now used). The result of this is a determination of the edge of the lesion with a high level of accuracy to the edge being one pixel deep.

The two-stage process described advantageously provides very efficient image processing. The ‘seeded growing’ can be relatively slow (order of minutes rather than seconds) for high resolution images from the camera that have not been cropped. By starting on a lower resolution image the approximate edge is first determined which when allows to crop the high resolution image close to the lesion edge and thereby reduce the seeded growing processing time significantly.

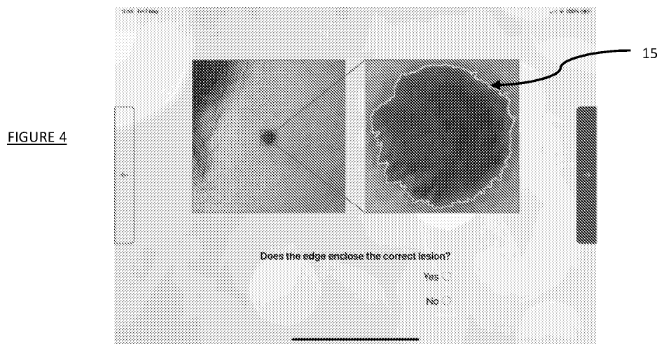

The refined, higher accuracy edge, is then displayed to the user as shown in FIG. 4. (This Figure shows a lesion different to that shown in FIGS. 2 and 3, but which has undergone the same processing as the lesion shown in those Figures.) The original image is shown as well as the cropped image showing the boundary 15. The user is prompted to provide an input which is indicative of whether the boundary which has been calculated properly encloses the lesion. If the user provides an answer in the affirmative then the boundary is determined as sufficiently accurate for any subsequent analysis thereon. This analysis, which is not part of the present disclosure, may include calculating a measure of irregularity of the boundary, which, in broad terms, may provide a measure of the likelihood of the lesion being malignant. If the analysis is beyond a threshold which is indicative of the lesion not being malignant, then this can be reported. Based on the analysed characteristics of the determined boundary, it can be determined if malignancy can be ruled out. Otherwise, the user is recommended to seek advice from a clinician. The further analysis which may be performed on the determined boundary may also include (in addition or alternatively to boundary irregularity) analysis on the skin (and one or more characteristics thereof) which is contained within the boundary that has been accurately determined. This may include analysing colour, texture, and/or variability of the same. AI may be used beneficially in whole or in part for such further analysis. Said further analysis may include a diagnostic analysis.

In the above embodiment, a deterministic approach is advantageously implemented. This means that the same boundary is determined if the process is repeated for the same lesion. This is unlike known edge determination approaches in which a different edge may be determined on different instances of processing the same lesion.

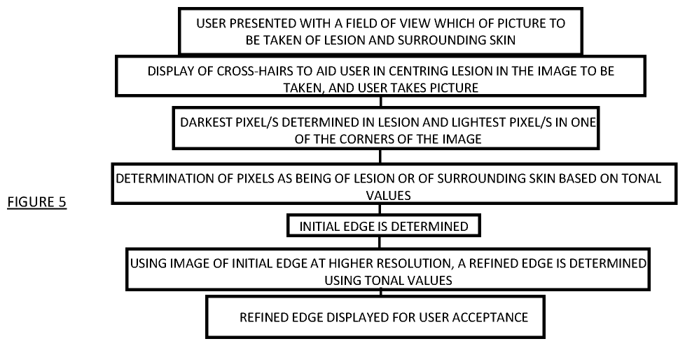

FIG. 5 is a flow chart which summarises the main steps implemented by the software.

1. Machine-readable instructions saved on non-transient memory for execution by data processor, for processing a captured image, which includes a lesion, to determine a boundary of the lesion, which instructions when executed by the data processor implement at least the following steps:
displaying the image, which image includes a lesion under investigation and includes a portion of skin adjacent to the lesion;
determining a lesion skin reference tone within the image, which comprises selecting one or more representative pixels within the lesion;
determining an adjacent skin reference tone of skin adjacent to the lesion which is also in the image, which comprises selecting one or more representative pixels not of the lesion;
analyzing tone levels of pixels within the image individually and/or in groups to determine said pixels of the image as either lesion tone pixels or adjacent skin tone pixels, which analysis evolves progressively away from each of the one or selected representative pixels of the lesion and the one or more selected representative pixels not of the lesion, respectively; and
determining within the image a boundary of where lesion skin tone pixels meet adjacent skin tone pixels, wherein the instructions are configured to implement a two-stage process to determine the boundary, in which a first stage comprises calculating an initial boundary at a first image resolution and then calculating a refined boundary at a second image resolution, wherein the second image resolution is higher than the first image resolution.

2. The machine-readable instructions of claim 1 which are arranged to determine a darkest level of lesion skin tone.

3. The machine-readable instructions of claim 2 which are arranged to determine a darkest level of lesion skin tone by identifying at least one pixel from within the lesion region is determined as being the pixel from a subset which has substantially the darkest level of tone of the lesion.

4. The machine-readable instructions of claim 3 in which the set of pixels is at or proximal to a central region of the image.

5. The machine-readable instructions of preceding claim 1 which are arranged to determine an adjacent skin reference tone of skin adjacent to the lesion by identifying at least one pixel which is away from the lesion and within the image.

6. The machine-readable instructions of claim 5 in which the at least one pixel is analyzed in each of multiple locations in the image.

7. The machine-readable instructions of claim 1 which are arranged to generate an initial determined boundary of the lesion.

8. The machine-readable instructions of claim 7 which are configured to sequentially analyze the tonality of pixels within the image, starting from at least two different locations with at least one being in the lesion and one being outside of the lesion.

9. The machine-readable instructions of claim 7 in which the initial boundary is determined as where lesion tone pixels neighbor adjacent skin tone pixels.

10. The machine-readable instructions of claim 7 in which are configured to determine a refined lesion boundary which has a higher accuracy than the initial boundary.

11. The machine-readable instructions of claim 10 which are configured to determine the refined lesion boundary by using a higher resolution version of the image as compared to a resolution of image used to determine the initial boundary, and by using the initial boundary.

12. The machine-readable instructions of claim 11 which are such as to map the initial boundary onto the higher resolution of the image.

13. The machine-readable instructions of claim 12 which are configured to generate a cropped version of the image which includes the lesion.

14. The machine-readable instructions of claim 13 which are configured to generate the cropped image once the lesion has been mapped onto the higher resolution version.

15. The machine-readable instructions of claim 13 in which the cropped version of the image is generated using location of the initial boundary.

16. The machine-readable instructions of claim 11 which are such as to generate the cropped image as having its major portion depicting the lesion and a minor portion depicting skin surrounding the lesion.

17. The machine-readable instructions of claim 11 which are such as to apply pixel tone analysis to the cropped image and thereby determine the refined boundary.

18. (canceled)

19. The machine-readable instructions of claim 1 which are configured to generate centering graphics in a graphic user interface to guide a user to center the lesion in the image to be taken.

20. The machine-readable instructions of claim 19 which are arranged to provide the centering graphics overlaid on a magnified part of the field of view of a camera.

21. The machine-readable instructions of claim 1 configured to generate an output an image which includes a determined lesion boundary and the lesion, and the determined lesion boundary is displayed as superimposed on the image of the lesion.

22. The machine-readable instructions of claim 21 configured to prompt the user review the image, and provide an input in relation to an assessment of a perceived accuracy of the boundary.

23. The machine-readable instructions of claim 1 which are configured to generate a lower resolution version of the captured image, which lower resolution image is used to determine an initial lesion boundary.

24. A user device which is loaded with the machine-readable instructions of claim 1.
